(19) United States
(12) Patent Application Publication
(10) Pub. No.: US 2025/0332346 A1
(43) Pub. Date: Oct. 30, 2025

(54) IMPROVED DELIVERY DEVICES

(71) Applicant: Boston Scientific Scimed, Inc., Maple Grove, MN (US)

(72) Inventors: Katie Knowles, Providence, RI (US); Roma Taranto, Ashland, MA (US); Hannah Hutton, Murfreesboro, TN (US); Sicheng Pang, Jing'an District (CN); Dianne Hernandez Huezo, Lynn, MA (US); Patrick Nicolas, Mansfield, MA (US); Benjamin Cleveland, Bellingham, MA (US); Susannah Howe, Florence, MA (US); Jonathan J. Lund, Glencoe, MN (US)

(73) Assignee: Boston Scientific Scimed, Inc., Maple Grove, MN (US)

(21) Appl. No.: 19/188,967

(22) Filed: Apr. 24, 2025

Related U.S. Application Data
(60) Provisional application No. 63/638,548, filed on Apr. 25, 2024.

(51) Int. Cl. A61M 5/19; A61M 5/315; A61M 39/00; A61M 39/10

(52) U.S. Cl. CPC A61M 5/19; A61M 5/31591; A61M 39/10; A61M 2039/0027; A61M 2039/1077; A61M 2202/0007

(57) ABSTRACT

A delivery device comprises a first syringe, a second syringe, a needle or a tube, and a Y-connector that includes at least one access port.

### CROSS-REFERENCE TO RELATED APPLICATIONS

This application claims the benefit of U.S. Provisional Patent Application Ser. No. 63/638,548 filed on Apr. 25, 2024, the disclosure of which is incorporated herein by reference.

### FIELD

The present disclosure relates to improved delivery devices, systems and methods. For instance, the improved delivery devices, systems, and methods may be employed to deliver or inject hydrogels, among other possible materials. Hydrogels are useful, for example, in various medical applications.

### BACKGROUND

Hydrogels with shear-thinning properties are known which undergo a reversible gel-sol transition upon the application of shear stress. J. Pushpamalar, et al., “Development of a polysaccharide-based hydrogel drug delivery system (DDS): An update.” Gels, 2021, 7(4), p.153. Shear-thinning hydrogels are increasingly used in drug delivery systems as a result of their ability to conform to the shape of an injection cavity, which maximizes contact with targeted tissue for localized drug delivery. Id. Hydrogels with shear-thinning properties have been reported to provide smooth injection without injection needle clogging (e.g., partial or complete flow restriction), with the hydrogels returning to their original properties once mechanical load (shear stress) is removed. M. H. Chen, et al., “Methods to assess shear-thinning hydrogels for application as injectable biomaterials.” ACS Biomater. Sci. Eng. 2017, 3, 3146-3160. Hydrogels assembled by physical crosslinking of polyvinyl alcohol (PVA) and polyvinyl pyrrolidone (PVP) and containing doxorubicin have been noted to be able to deform under high shear and subsequently retain their original shape upon the removal of the high shear, demonstrating both shear-thinning and self-healing properties. N. K. Prasad, et al., “Discerning the self-healing, shear-thinning characteristics and therapeutic efficacy of hydrogel drug carriers migrating through constricted microchannel resembling blood microcapillary,” Colloids Surf. A Physiochem. Eng. Asp. 2021, 626, 127070. FIG. 1 on page 5 of J. Pushpamalar, et al., schematically illustrates the shear-thinning and self-healing properties of a doxorubicin-loaded poly(vinyl alcohol)/poly(vinyl pyrrolidone) hydrogel. A shear-thinning hydrogel containing gelatin and laponite for localized drug delivery and further containing chitosan and poly(N-isopropylacrylamide-co-acrylic acid) particles to render the hydrogel pH-responsive has also been reported. S. Gharaie, et al., “Smart shear-thinning hydrogels as injectable drug delivery systems.” Polymers, 2018, 10(12), p.1317.

SpaceOAR™ is a rapid crosslinking hydrogel that polymerizes in vivo and is based on a multi-arm polyethylene glycol (PEG) polymer functionalized with succinimidyl glutarate as activated end groups which further react with trilysine to form crosslinks. This product has been used clinically in prostate cancer therapy.

### BRIEF SUMMARY

A first example is a delivery device comprising: a first syringe comprising a first plunger and a first syringe barrel that terminates in a first connector and, a second syringe comprising a second plunger and a second syringe barrel that terminates in a second connector; a needle or a tube; and a Y-connector, wherein the Y-connector includes at least one access port.

Alternatively or additionally to any of the examples herein, in another example, wherein the at least one access port includes a proximal access port or a common access port.

Alternatively or additionally to any of the examples herein, in another example, wherein the at least one access port includes both a proximal access port and a common access port.

Alternatively or additionally to any of the examples herein, in another example, wherein the proximal access port is located at a proximal end of a common branch of the Y-connector.

Alternatively or additionally to any of the examples herein, in another example, wherein at least a portion of the proximal access port is located along a longitudinal axis of the delivery device.

Alternatively or additionally to any of the examples herein, in another example, wherein the proximal access port is configured to permit a clearance instrument to extend through the proximal access port at least into a common lumen of the common branch.

Alternatively or additionally to any of the examples herein, in another example, wherein at least a portion of the common access port is located along a longitudinal axis of the Y-connector.

Alternatively or additionally to any of the examples herein, in another example, wherein the common access port is an elongate recess in the common branch, and wherein the elongate recess is configured to receive a removable mixer module.

Alternatively or additionally to any of the examples herein, in another example, the delivery device further comprising a removable mixer module that is removably disposed in the common access port.

Alternatively or additionally to any of the examples herein, in another example, wherein the removable mixer module includes a handle.

Alternatively or additionally to any of the examples herein, in another example, wherein the delivery device includes a movable mixer module including a plurality of static mixers coupled thereto, wherein the movable mixer module is configured to position an individual static mixer, of the plurality of static mixers, in a common lumen of a common branch of the Y-connector.

Alternatively or additionally to any of the examples herein, in another example, wherein the movable mixer module is movable between a first position and a second position, and wherein a first static mixer of the plurality of static mixers is in the common lumen when the movable mixer module is in the first position, and wherein a second static mixer of the plurality of static mixer is in the common lumen when the movable mixer module is in the second position.

Alternatively or additionally to any of the examples herein, in another example, wherein the at least one access port includes a proximal access port, and wherein the delivery device further comprises a third syringe comprising a third plunger and a third syringe barrel, and wherein the third syringe is fluidically coupled to a proximal access port.

Alternatively or additionally to any of the examples herein, in another example, wherein the third syringe includes a fluid.

Alternatively or additionally to any of the examples herein, in another example, wherein the delivery device includes a plurality of static mixers that are fixed relative to the delivery device, and wherein the delivery device is configured to: select a first static mixer for a flow path of the delivery device extend therethrough in a first configuration; and select a second static mixer for the flow path of the delivery device extend therethrough in a second configuration.

Another example is a material injection system comprising; delivery device comprising: a first syringe comprising a first plunger and a first syringe barrel that terminates in a first connector; a second syringe comprising a second plunger and a second syringe barrel that terminates in a second connector; a needle or a tube; and a Y-connector that comprises a first branch lumen having a first end and a second end, a second branch lumen having a first end and a second end, and a common branch that comprises a common lumen having a first end and a second end, the first end of the first branch lumen and the first end of the second branch lumen in fluid communication with the first end of the common lumen, the second end of the first branch lumen terminating at a connector which is configured to connect with the first connector, the second end of the second branch lumen terminating at a connector which is configured to connect with the second connector, and the second end of the common lumen terminating at a connector configured to connect to a connector of the needle or the tube, wherein the Y-connector includes at least one access port; and a clearance instrument.

Alternatively or additionally to any of the examples herein, in another example, wherein the clearance instrument comprises an elongate rod or a wire.

Alternatively or additionally to any of the examples herein, in another example, wherein the clearance instrument is stored in a clearance instrument storage device, and wherein the clearance instrument storage device is coupled to or is removably coupled to the delivery device.

Another example a method of material injection, comprising: simultaneously pressing a first plunger and a second plunger into a first syringe barrel of a first syringe and a second syringe barrel of a second syringe, respectively, to cause a portion of a first solution in the first syringe and a second solution in the second syringe to form an injectable material that is injected; pausing pressing the first plunger and second plunger prior to injection of all of the first solution and the second solution; dislodging, via a peripheral access port, a common access port, a bypass port, or any combination thereof, a clog formed of a portion of the injectable material that remains along an injection path of a delivery device, wherein dislodging the clog comprises passing a clearance instrument through the peripheral access port, the common access port, the bypass port, or any combination thereof, along at least a portion of a flow path of the delivery device; and simultaneously pressing the first plunger and the second plunger into the first syringe barrel and the second syringe barrel, respectively, to cause at least part of a remaining portion of the first solution in the first syringe and at least part of the second solution in the second syringe to form an injectable material.

Alternatively or additionally to any of the examples herein, in another example, wherein the injectable material is an injectable hydrogel.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

FIG. 1 schematically illustrates a delivery device, in accordance with an embodiment of the present disclosure.

FIG. 2 schematically illustrates a Y-connector, in accordance with an embodiment of the present disclosure.

FIG. 3 schematically illustrates an exploded view of components of the Y-connector, in accordance with an embodiment of the present disclosure.

FIG. 4A schematically illustrates a removable mixer module, in accordance with an embodiment of the present disclosure.

FIG. 4B schematically illustrates another removable mixer module, in accordance with an embodiment of the present disclosure.

FIG. 5A schematically illustrates a Y-connector including a movable mixer module in a first configuration, in accordance with an embodiment of the present disclosure.

FIG. 5B schematically illustrates the Y-connector of FIG. 5A with the movable mixer module in a second configuration, in accordance with an embodiment of the present disclosure.

FIG. 6A schematically illustrates a Y-connector including a movable mixer module in a first configuration, in accordance with an embodiment of the present disclosure.

FIG. 6B schematically illustrates the Y-connector of FIG. 6A with the movable mixer module in a second configuration, in accordance with an embodiment of the present disclosure.

FIG. 7A schematically illustrates a Y-connector including a fixed mixer module with the Y-connector in a first configuration, in accordance with an embodiment of the present disclosure.

FIG. 7B schematically illustrates the Y-connector of FIG. 7A with the Y-connector in a second configuration, in accordance with an embodiment of the present disclosure.

FIG. 8A schematically illustrates a valve in a first configuration, in accordance with an embodiment of the present disclosure.

FIG. 8B schematically illustrates the valve of FIG. 8A in a second configuration, in accordance with an embodiment of the present disclosure.

FIG. 9A schematically illustrates a delivery device in a first configuration, in accordance with an embodiment of the present disclosure.

FIG. 9B schematically illustrates a delivery device in a second configuration, in accordance with an embodiment of the present disclosure.

FIG. 9C schematically illustrates a delivery device in a third configuration, in accordance with an embodiment of the present disclosure.

FIG. 9D schematically illustrates a delivery device in a fourth configuration, in accordance with an embodiment of the present disclosure.

FIG. 9E schematically illustrates a delivery device in a fifth configuration, in accordance with an embodiment of the present disclosure.

FIG. 9F schematically illustrates a delivery device in a sixth configuration, in accordance with an embodiment of the present disclosure.

FIG. 9G schematically illustrates a delivery device in a seventh configuration, in accordance with an embodiment of the present disclosure.

FIG. 10 schematically illustrates a delivery device including a fluid reservoir manifested in the form of a syringe, in accordance with an embodiment of the present disclosure.

FIG. 11 illustrates an elongate rod in the proximal access port and extending longitudinally along a fluid flow path within the delivery device.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

The present disclosure relates to improved delivery devices, systems, and methods. Potential benefits associated with the present disclosure include mitigation of any issues (e.g., unintended clogging of a delivery device and/or clogging of an injection needle) associated with previous injection approaches. As used herein, clogging refers to the partial or complete obstruction of a flow path within a device such as a flow path within a delivery device and/or injection needle. For instance, previous approaches which may require a continuous or near continuous delivery or injection of material such as hydrogel to avoid clog formation. Otherwise, if a continuous or near continuous injection is not maintained clogs may develop in the delivery device before a desired volume of material has been delivered to the patient. In such instances, the user may become unable to finalize placement of the material due to the clog formation. Additionally, such approaches may prevent the user from customizing a shape and/or placement location of the material (e.g., hydrogel) but may instead limit the material injection to a given location (e.g., to ensure a continuous or near continuous injection is maintained). As such, a resultant shape of the material may be dictated primarily or entirely by the individual patient anatomy at the given position.

Thus, it may be desirable to permit a user to stop and start an injection or delivery of a material at the given location and permit the user to subsequently start another injection of the remaining material in the delivery device at a different location. Accordingly, the delivery devices herein are configured to permit any claims (e.g., hydrogel clogs) formed within the delivery device and/or an injection needle, catheter, or other injection device coupled to the delivery device to be unclogged. As such, the delivery devices herein can provide the opportunity for a user to user to stop and start an injection at the given location, dislodge any material (e.g., hydrogel) clogs, and permit the user to subsequently start another injection of the remaining material in the delivery device at a different location. As such, the delivery devices herein can permit the user to customize placement or “sculpting” of a material such as hydrogel, and yet can also mitigate issues (e.g., clog formation) that are typically associated with delivery of various material by delivery devices. While references are made herein to hydrogel delivery devices suitable for hydrogel injection, the present disclosure is not so limited but instead may be employed with various other types of two-part materials such as epoxies, etc.

As mentioned, in some embodiments that delivery devices may be employed with hydrogels. As used herein, a hydrogel refers to a water-containing three-dimensional network of crosslinked polymers. In some embodiments, the injectable hydrogels are shear-thinning and self-assembling injectable hydrogels. The shear-thinning properties of such hydrogels allow for efficient injectability, as the hydrogels exhibit viscous flow under shear. In some embodiments, the injectable hydrogels exhibit yielding behavior. For example, after being subjected to a threshold yield strain, the injectable hydrogels may exhibit sharp decreases in storage and loss moduli, which decreases in moduli are recovered at low strains upon cessation of shear. The self-assembling properties of such hydrogels (also referred to as self-healing properties) allow for re-formation and stabilization of the hydrogel when the shear stress is removed. As used herein, self-assembly and self-healing refer to the spontaneous formation of new bonds within a material after old bonds within the material are broken. The injectable hydrogels of the disclosure may include a carrier fluid. The carrier fluid in the injectable hydrogels may be water. The water may be provided in the form of ultrapure water, water for injection, saline, phosphate buffered saline, or high-ion-content water. In some embodiments, the injectable hydrogels contain between 0.25 weight percent (wt %) or less and 30 wt % or more water, for example, ranging anywhere from 0.25 to 0.5 to 1 to 2.5 to 5 to 10 to 20 to 30 wt %.

In some embodiments, some or all of the components of a delivery device (e.g., a hydrogel delivery device) may be configured to undergo sterilization (e.g., sterilization via steam ultraviolet, gamma radiation, and/or x-ray exposure, etc.). In some embodiments, each of the components of the delivery device 100 may be configured to withstand steam sterilization. For instance, some or all of the components may be formed of materials that are suitable to withstand steam sterilization (e.g., retain their physical form and/or properties during and subsequent to undergoing steam sterilization). Examples of suitable materials (e.g., which retain their physical form) include glass, polycarbonate, polypropylene, rubber, and/or nylon, among other suitable materials.

In various embodiments relating to hydrogels, the injectable hydrogels comprise (a) one or more types of hydrogen bond donors, (b) one or more types of hydrogen bond acceptors, and (c) water. Such hydrogels comprise hydrogen-bond-based crosslinks which dissociate when a shear stress is applied, and which spontaneously self-assemble when the shear stress is removed. Such disassociation may occur, for example, when a shear stress is applied during injection from a syringe. Upon dissociation of the hydrogen-bond-based crosslinks, the hydrogel becomes a viscous liquid that can be transported to a target site though a suitable delivery device, such as a tube (e.g. catheter/microcatheter) or a needle. Once delivered to the target site and the shear stress diminishes, the hydrogen bonds spontaneously re-associate (i.e., self-assemble), reforming the hydrogel at the target site. The transformation of the viscous liquid back into a hydrogel results in improved material retention and mechanical properties.

The injection devices and systems herein may also include a Y-connector that is configured to combine and mix the contents of the first and second syringes into a combined stream which can then be injected into the patient, for example, through a needle or a tube. For example, the Y-connector may include a first branch lumen having a first end and a second end, a second branch lumen having a first end and a second end, and a common lumen having a first end and a second end. The first end of the first branch lumen and the first end of the second branch lumen may be in fluid communication with the first end of the common lumen at a merge point. The second end of the first branch lumen may terminate at a connector (e.g., a Luer connector) which is configured to connect with a complementary connector of the first syringe barrel, the second end of the second branch lumen may terminate at a connector (e.g., a Luer connector) which is configured to connect with a complementary connector of the second syringe barrel, and the second end of the common lumen may terminate at a connector (e.g., a Luer connector) which is configured to connect with a complementary connector of a needle or a tube (e.g., a Luer connector).

The injection devices and systems herein may optionally further include one, two or all three of the following: a syringe holder that is configured to hold the first and second syringe barrels in a fixed relationship, a plunger cap that is configured to hold the first and second plungers in a fixed relationship, and a vial adapter for providing fluid communication between the syringe barrel and the vial. Such a vial adaptor may include a spike, which is configured for the puncturing an elastomeric closure of the vial containing the iodinated polymer composition, thereby accessing the interior of the vial, and a connector (e.g., Luer connector), which is configured for attachment to the first syringe barrel.

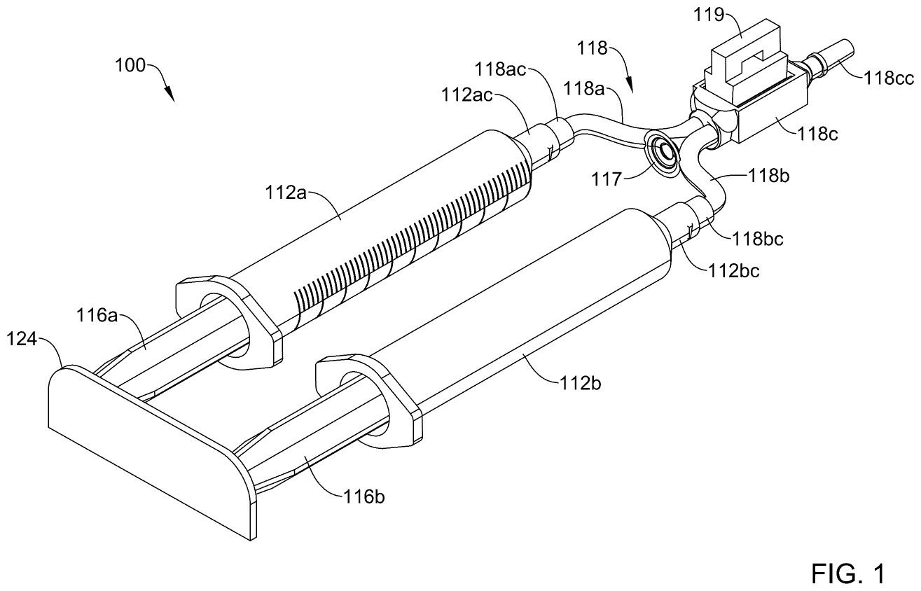

FIG. 1 schematically illustrates a delivery device 100, in accordance with an embodiment of the present disclosure. The delivery device 100 includes a connector 112ac of the first syringe barrel 112a (e.g., containing a buffered precursor solution) is connected to a first connector 118ac of a first branch 118a of a Y-connector 118, and a connector 112bc of the second syringe barrel 112b (e.g., containing a buffered accelerant solution) is connected to a second connector 118bc of a second branch 118b of the Y-connector 118. Also shown are a first plunger 116a that is movable in the first syringe barrel 112a, a second plunger 116b that is movable in the second barrel 112b, a syringe holder (not illustrated) configured to hold the first and second syringe barrels 112a, 112b, in a fixed relationship and a plunger cap 124 configured to hold the first and second plungers 116a, 116b in a fixed relationship. After the first syringe barrels 112a, 112b are attached to the Y-connector, the syringe holder (not illustrated) is attached to the first and second syringe barrels 112a, 112b, and the plunger cap 124 is attached to the first and second plungers 116a, 116b, as is conventionally known. A needle or tube (not shown) is attached to and/or is integral with the common branch connector 118cc, as is conventionally known.

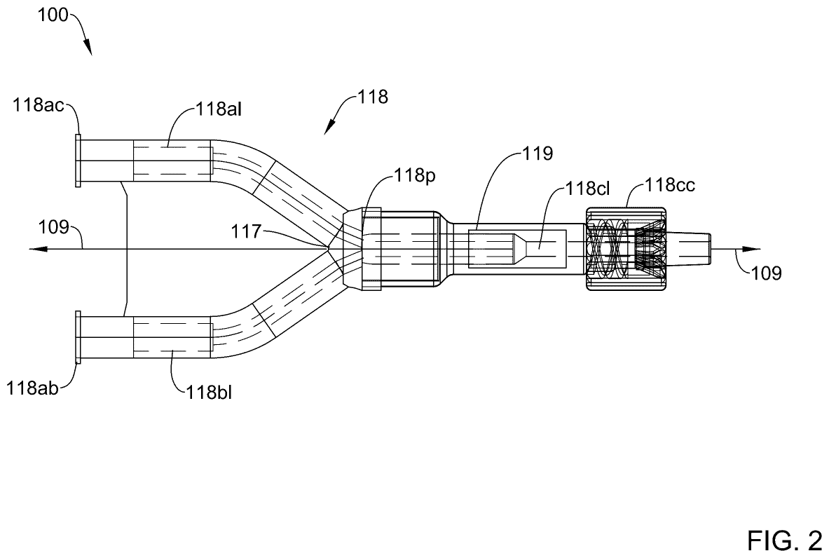

During operation, the first plunger 116a is pressed into the first syringe barrel 112a, forcing a first solution (e.g., the buffered precursor solution) through the lumen of the first branch 118a of the Y-connector 118. Simultaneously, the second plunger 116b is pressed into the second syringe barrel 112b, forcing a second solution (e.g., the buffered accelerant solution) through lumen of the second branch 118b of the Y-connector. The first solution (e.g., the buffered precursor solution) and the second solution (e.g., the buffered accelerant solution) meet and mix at a merge point (e.g., merge point 118p as illustrated in FIG. 2), with the resultant mixture moving along the lumen of the common branch 118c and exiting the Y-connector 118 at the common branch connector 118cc. For instance, combining a buffered accelerant solution with the buffered precursor solution can increase the pH of the resulting mixture, causing crosslinking e.g., between an iodinated polymer and the polyamine, which leads to the formation of a hydrogel.

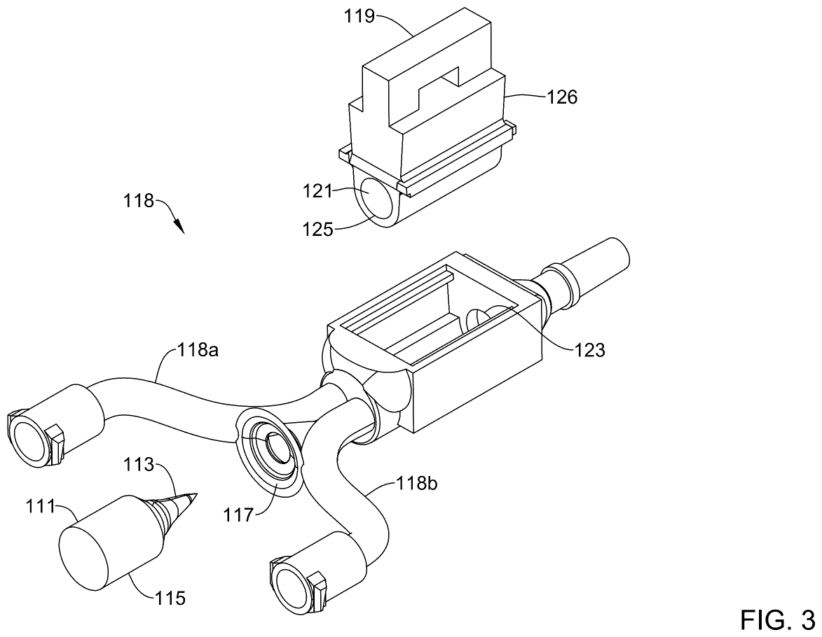

FIG. 2 schematically illustrates a Y-connector 118, while FIG. 3 schematically illustrates an exploded view of components of the Y-connector 118. The Y-connector 118 includes a common branch 118c having an optional common branch connector 118cc. A region including a merge point 118p is shown, where the lumens of the first branch 118a and the second branch 118b merge into the lumen of the common branch 118c. The Y-connector 118 can include a first branch lumen 118al terminating at a first Luer connector 118ac, a second branch lumen 118bl terminating at a second Luer connector 118bc and a common lumen 118cl terminating at a first Luer connector 118ac. The first branch lumen 118al and the second branch lumen 118bl, along with the other components herein, can be formed using tubing and/or other types of structures having the same or differing diameters (e.g., differing hypotubes having various inside diameters). While described as including Luer connectors, the use of other types of connectors is possible.

As illustrated in FIG. 3, the Y-connector 118 can include at least one access port. For instance, the Y-connector 118 can include a proximal access port 117, common access port 123, or both. For example, the Y-connector 118 can include both a proximal access port 117 and a common access port 123. In such instances, the Y-connector 118 can include at least two access ports at two different locations in the Y-connector 118, as illustrated in FIG. 3.

The proximal access port 117 can provide access to a proximal end portion of the common lumen 118cl. The common access port 123 can be centrally or substantially centrally located in the common branch 118c of the Y-connector 118 and the proximal access port 117 can be located on a proximal end of the common branch 118c, as illustrated in FIG. 3. The common access port 123 can be an elongate recess in the common branch connector, as shown in FIG. 3. The common access port 123 can provide access to a central or substantially central portion of the common lumen 118cl. The common access port 123 can be configured to receive a removable mixer module 119 such that the removable mixer module is at least partially in the common access port 123, as illustrated in FIG. 3.

The proximal access port 117 can be located at an intersection between the first branch 118a and the second branch 118b. Stated differently, the proximal access port 117 can be located along a longitudinal axis (e.g., the longitudinal axis 109 as illustrated in FIG. 2) of the delivery device 100, as illustrated in FIG. 2. However, in some instances, the proximal access port 117 can be located elsewhere, for instance, such as being located along the common branch 118c and/or the common branch 118c and/or being located coaxial with the longitudinal axis 109.

The proximal access port 117 can be manifested as an orifice or valve. For instance, the proximal access port 117 can be configured as an orifice and the delivery device 100 can include a removable plug 111 configured to fluidically seal the orifice, as detailed herein. In such instances, the removable plug 111 can be selectively removed from the orifice to permit a clog formed in the delivery device to be removed, as detailed herein. For instance, the removable plug can include a body region 115 having a substantially circular cross-section and an insertion region 113 that extends distally from the body region 115 and is tapered or otherwise configured to fluidically seal the proximal access port 117 when the removable plug is in the proximal access port 117. However, other configurations are possible. For instance, in some embodiments the proximal access port 117 can be manifested as a valve, namely a one-way valve. The one-way valve can be configured to permit a clog formed in the delivery device to be removed (e.g., by way of insertion of a clearance instrument through the one-way valve and into at least a volume of the common branch 118c). Y et, the one-way valve can fluidically seal the delivery device 100 in the absence of the insertion or presence of the clearance instrument in the seal, as detailed herein. That is, the access port 117 can alone (e.g., when manifested as a one-way value), or in conjunction with a removable plug (e.g., when manifested as an orifice in the proximal end of the Y-connector), be configured to fluidically seal the proximal end of the Y-connector (e.g., such that hydrogel is only permitted to be ejected from a distal end of the delivery device 100).

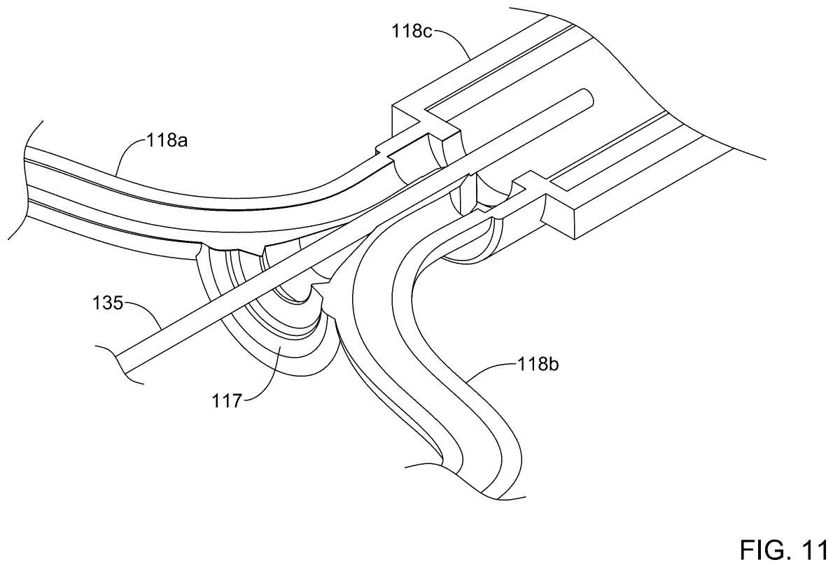

The proximal access port 117 can be configured (e.g., with a shape and/or size) to permit insertion and/or removal of a clearance instrument (e.g., clearance instrument 135 Fig 11) such as a clearance rod into at least the common branch 118c. For instance, the proximal access port 117 can have a substantially circular or substantially oval cross-section (extending radially from the longitudinal axis of the delivery device 100), among other possible configurations. Similarly, the clearance instrument (e.g., clearance instrument 135, as detailed herein) can be configured (e.g., with as shape or diameter) that substantially conforms to but is less than an intraluminal diameter of the common branch 118c. Thus, insertion or removal of the clearance instrument can physically dislodge a clog formed in the common branch 118c (e.g., can cause the clog to by physically moved distally along the common branch 118c and out of the branch connector 118cc. For example, the clearance instrument can have a substantially circular or substantially oval cross-section. While various embodiments herein describe the clearance instrument 135 as being inserted into the proximal access port 117 and extending distally therefrom to dislodge any clogs along a flow path of a delivery device, in some embodiments the clearance instrument 135 can be inserted in a distal end of the delivery device and can extend proximally out of the proximal access port 117 to dislodge any clogs along a flow path of the delivery device.

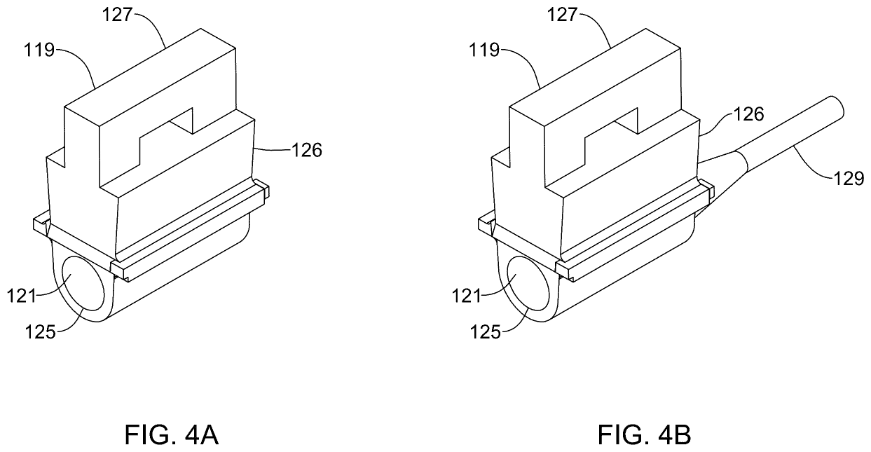

FIG. 4A schematically illustrates a removable mixer module 119. The removable mixer module 119 can include a body 126 with a handle 127 extending therefrom. The body 126 can include tapered portions and/or flanges that are configured to cause the removable mixer module 119 to friction fit in the common access port 123. For instance, some or all of the body 126 can be configured to be disposed in a volume of the common access port 123.

The body 126 can define a lumen 121 extending substantially longitudinally therethrough. For instance, the lumen 121 can extend from a proximal surface of the removable mixer module 119 to a distal surface of the removable mixer module 119. The lumen 121 can be configured to receive a mixer such as a static mixer 125. The static mixer 125 can be a ribbon mixer, an in-line mixer, and or any other suitable static mixer. In some cases, the static mixer 125 may instead be replaced with an active mixer that includes one or more moving parts, however this is not shown herein. The static mixer 125 may include a plurality of blades (not shown) that extend into a mixing chamber (not shown). The plurality of blades is stationary, providing a stop for a fluid injected into the mixing chamber, thereby causing the injected fluids to mix together within the mixing chamber. In some cases, there may be one type of fluid injected into the mixing chamber, such as saline, for example. In some cases, there may be more than one type of fluid injected into the mixing chamber, such as water and polyethylene glycol (PEG), for example.

FIG. 4B schematically illustrates another removable mixer module 119. The removable mixer module 119 of FIG. 4B is analogous to the removable mixer module 119 of FIG. 4A, but with the addition of the longitudinal protrusion 129 extending substantially longitudinally from the body 126 of the removable mixer module 119 of FIG. 4B. When disposed in the common access port 123, the longitudinal protrusion 129 can extend from the static mixer 125 along a longitudinal axis of the delivery device 100. For instance, the longitudinal protrusion 129 can extend distally from the static mixer 125 along the longitudinal axis of the delivery device 100. In some instances, the longitudinal protrusion 129 can be configured to remove clogs (e.g., hydrogel clogs) that are distal to the common access port 123 and located along a flow path of the delivery device 100. Thus, replacement of the removable mixer module 119 can remove any clogs that would otherwise be in the removable mixer module 119, and in such embodiments can also dislodge clogs that are distal to the static mixer 119.

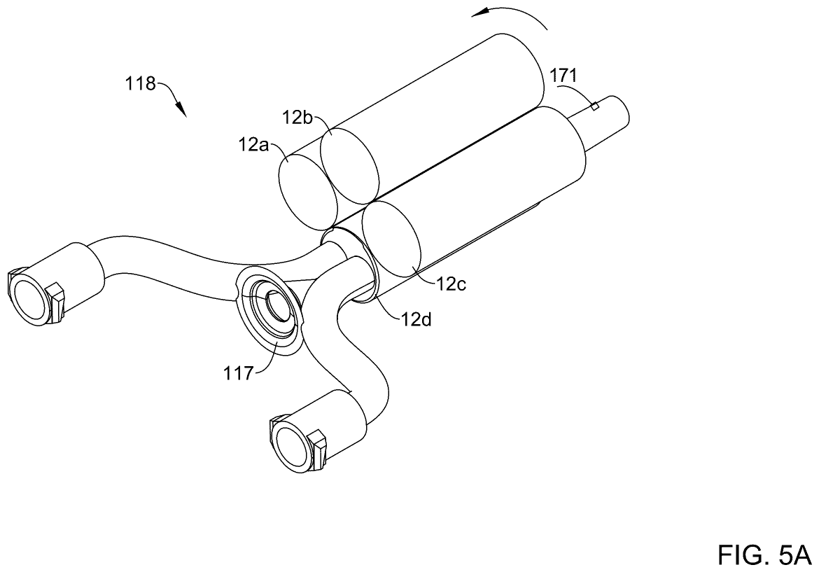

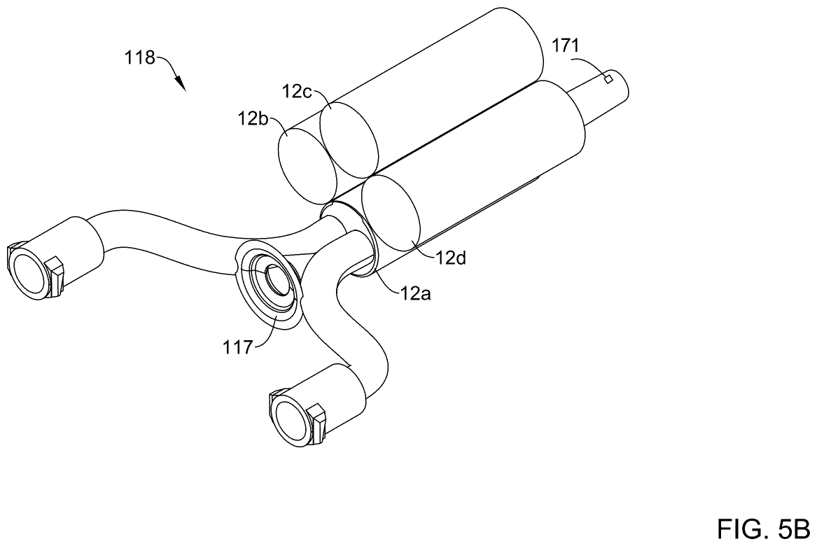

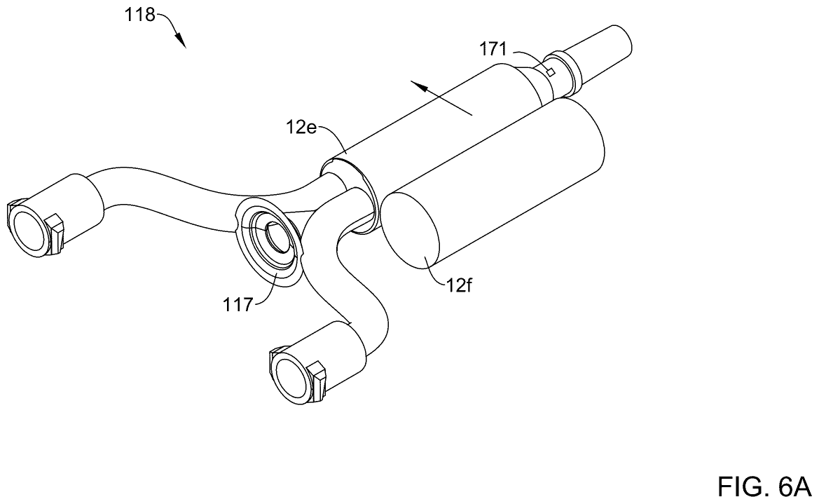

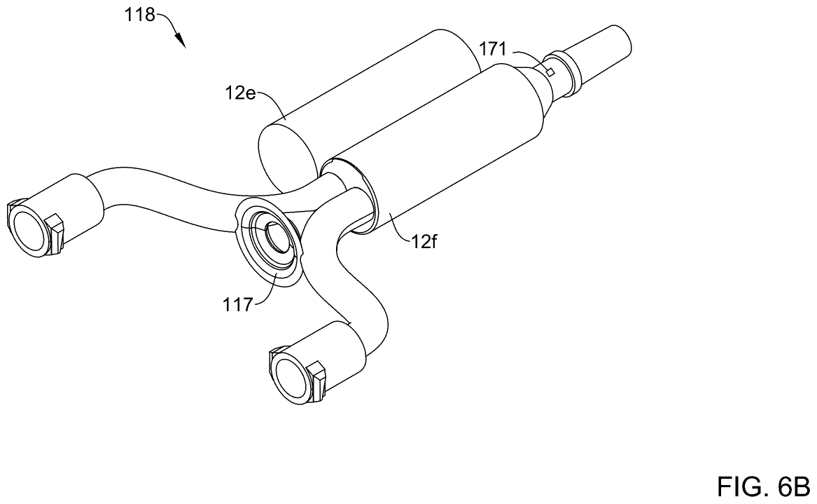

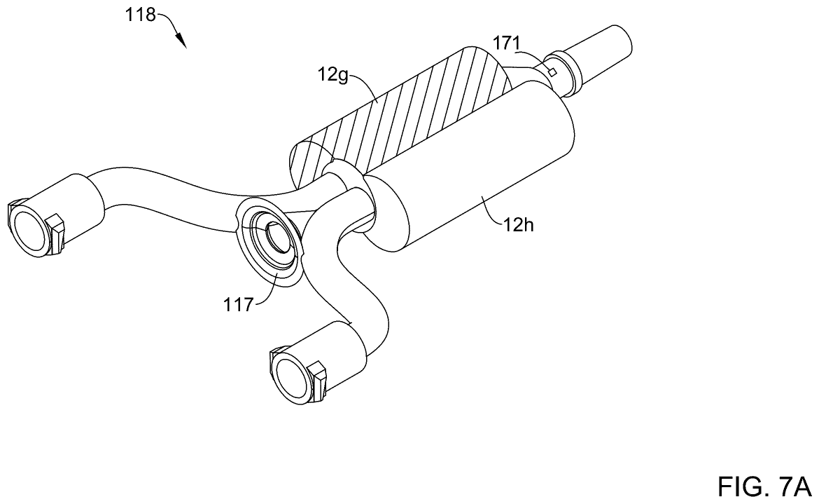

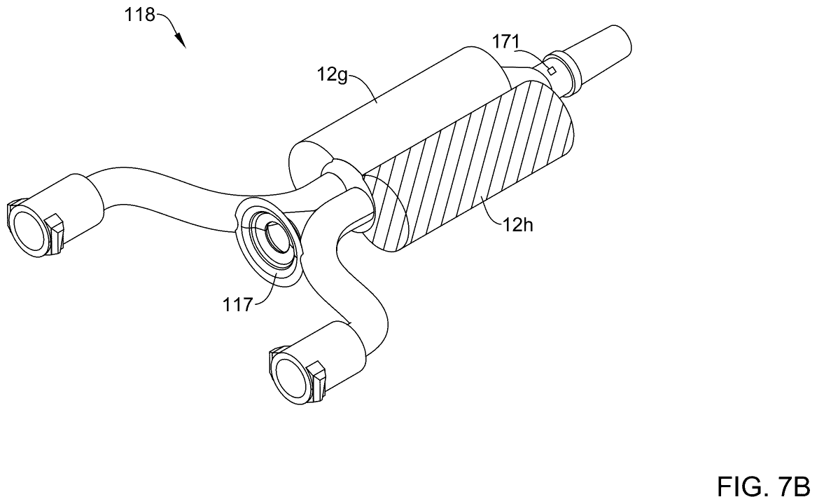

While FIGS. 1, 2, 3, and 4A-4B herein illustrate an individual static mixer as being disposed in the removable mixer module 119, other configurations are possible. For instance, FIGS. 5A-5B, 6A-6B, and 7A-7B illustrate various other configurations of the Y-connector that include a plurality of static mixers coupled thereto. As illustrated in FIGS. 5A-5B and 6A-6B, the plurality of static mixers can be included in a movable static mixer module that is movably coupled to the Y-connector such that an individual static mixer is disposed in a common lumen of the Y-connector. However, in FIGS. 7A-7B, each of the static mixers can be fixed relative to (non-movably coupled to) Y-connector and an individual static mixer of the plurality of static mixers can be selected (e.g., by way of movement of a valve, toggle, switch, and/or other mechanism) to have a fluid flow path extend therethrough (and not through any of the other static mixers). In any case, a flow path can extend through an individual static mixer when the Y-connector 118 is in a first position and can extend through a different static mixer when the Y-connector 118 is in a different position, as detailed herein.

FIG. 5A schematically illustrates a Y-connector 118 in a first configuration, in accordance with an embodiment of the present disclosure. As illustrated in FIG. 5A, the Y-connector 118 can include a plurality of static mixers 12a, 12b, 12c, and 12d coupled thereto. For instance, a plurality of static mixers 12a, 12b, 12c, and 12d can be movably coupled to the Y-connector 118 such that each of the static mixers 12a, 12b, 12c, and 12d can rotate about the Y-connector 118 such that any given one of the static mixers 12a, 12b, 12c, and 12d is disposed in the common lumen of the common branch at a given time. For instance, as illustrated in FIG. 5A a first static mixer 12a, a second static mixer 12b, and a third static mixer 12c can be disposed outside of the common lumen when the Y-connector 118 of FIG. 5A is in a first position, yet the fourth static mixer 12d can be disposed within the common lumen. In some embodiments, the Y-connector 118 can include a bypass access port 171. The bypass access port 171 can be included in addition to or in place of a peripheral access port. Similar to the peripheral access port, the bypass access port can be configured to permit a clearance instrument to be passed therethrough such that the clearance instrument passes distally along at least a portion of a flow path of the delivery device thereby dislodging any clogs along at least the portion of the flow path of the delivery device that is distal to the plurality of static mixers (e.g., the plurality of static mixers 12a, 12b, 12c, and 12d). That is, the bypass access port can permit a clearance instrument to “bypass” the plurality of static mixers and thereby restore the patency of at least a portion of the flow path of the delivery device that is distal to the plurality of static mixers.

FIG. 5B schematically illustrates the Y-connector 118 of FIG. 5A in a second configuration, in accordance with an embodiment of the present disclosure. Subsequent to the Y-connector of FIG. 5A being in the first position, the Y-connector 118 of FIG. 5A can be moved (e.g., rotated) to the second position. When in the second position, a different static mixer of the plurality of static mixers is disposed in the common lumen. For instance, as illustrated in FIG. 5B, the third static mixer can be disposed in the common lumen when the Y-connector of FIG. 5B is in the second configuration. Similarly, each of the other static mixers of the plurality of static mixers can be respectively disposed in the common lumen when the Y-connector 118 is in a different position. In this way, the Y-connector 118 permits at least four different static mixers to be employed within the injection devices herein. As such, the delivery devices (e.g., hydrogel delivery devices) herein can perform an initial material (e.g., hydrogel) injection or delivery, and can subsequently pause and restart material injection or delivery a plurality of times (e.g., three) times, as compared to other existing approaches the require continuous or near continuous material injection or delivery (e.g., at a fixed location).

FIG. 6A schematically illustrates a Y-connector 118 in a first configuration, in accordance with an embodiment of the present disclosure. Similar to the Y-connector 118 of FIGS. 5A-5B, the Y-connector 118 can include a plurality of static mixers 12e, 12f that are movably coupled to the Y-connector. For instance, the plurality of static mixers 12e, 12f can be movably coupled to the Y-connector 118 such that each of the static mixers 12e, 12f can translate (e.g., radially) relative to the Y-connector 118 such that any given one of the static mixers 12e, 12f, is disposed in the common lumen of the common branch at a given time. For instance, as illustrated in FIG. 6A a first static mixer 12e can be disposed in the common lumen when the Y-connector 118 is in a first position, yet the second static mixer 12f can be disposed outside the common lumen.

FIG. 6B schematically illustrates the Y-connector of FIG. 6A in a second configuration, in accordance with an embodiment of the present disclosure. Subsequent to the Y-connector of FIG. 6A being in the first position, the Y-connector 118 of FIG. 6A can be moved (e.g., translated) to the second position. When in the second position, a different static mixer of the plurality of static mixers is disposed in the common lumen. For instance, as illustrated in FIG. 6B, the second static mixer 12f can be disposed in the common lumen when the Y-connector of FIG. 6B is in the second position.

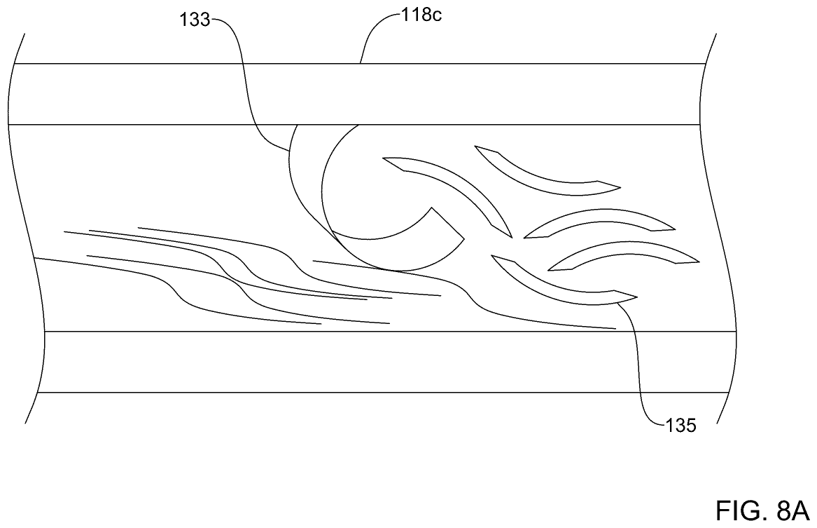

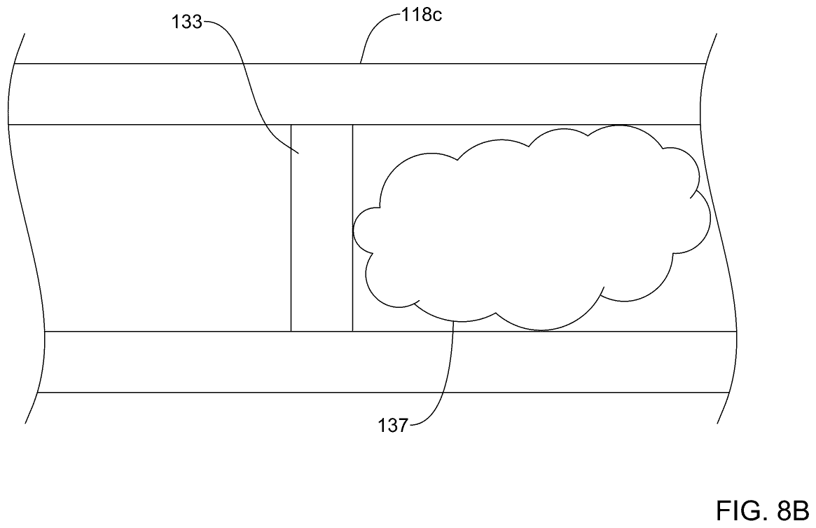

FIG. 7A schematically illustrates a Y-connector in a first configuration, in accordance with an embodiment of the present disclosure. FIG. 7B schematically illustrates the Y-connector of FIG. 7A in a second configuration, in accordance with an embodiment of the present disclosure. The delivery device can include a plurality of static mixers that are fixed relative to the delivery device, and wherein the delivery device is configured to select a first static mixer for a flow path of the delivery device extend therethrough in a first configuration; and select a second static mixer for the flow path of the delivery device extend therethrough in a second configuration, as described herein. That is, similar to the Y-connectors 118 of FIGS. 5A-5B and FIGS. 6A-6B, the Y-connector 118 of FIGS. 7A-7B can include a plurality of static mixers 12g, 12h that are movably coupled to the Y-connector 118. However, in FIGS. 7A-7B, each of the static mixers can be fixed to (non-movably coupled to) Y-connector and an individual static mixer of the plurality of static mixers can be selected (e.g., by way of movement of a valve, toggle, switch, and/or other mechanism) to have a fluid flow path extend therethrough (and not through any of the other static mixers). For instance, the flow path can extend through the first static mixer 12g, as represented in FIG. 7A. However, subsequent to activation of a switch, toggle, or other mechanism, the flow path can be altered to extend through the second static mixer 12h, as represented in FIG. 7B. In some embodiments, constituent flow could be directed to one static mixer or flow path at a time by way of regulators, valves, or switches that are manually actuated via a button, tab, or other mechanical, electromechanical, GUI, touchscreen, or other mechanism. In some embodiments, constituent flow could be directed by valves or switches that are automatically flipped by movement of the clearance instrument (e.g., a push/pull rod) during de-clogging of the delivery device. In some embodiments, constituent flow could be directed by valves whose movement is restricted by gel formation in the delivery device, such as the valves described with respect to FIGS. 8A and 8B.

While a given quantity of static mixers is illustrated in FIG. 5A-5B, 6A-6B, and/or 7A-7B, the quantity of static mixers in FIG. 5A-5B, 6A-6B, and/or 7A-7B can be varied (e.g., increased or decreased). The Y-connectors 118 in FIG. 5A-5B, 6A-6B, and/or 7A-7B can include various mechanisms (not shown) such as a housing, a pin, a pivot, a pawl, a lever, and/or other mechanism that are configured to movably couple the plurality of static mixers in FIG. 5A-5B, 6A-6B, and 7A-7B to the Y-connectors 118 therein.

FIG. 8A schematically illustrates a valve 133 in a first configuration, while FIG. 8B schematically illustrates the valve 133 in a second configuration. The valve 133 (represented as a movable sheet of material) can be formed of various types of valves such as various types of valves that can stop fluid flow (e.g., stop fluid flow in at least one direction). When in the first position, the valve 133 can permit fluid flow (represented as element 135) distally along a flow path extending through the common branch 118c (e.g., through a common lumen in the common branch 118c), as illustrated in FIG. 8A. For instance, the valve 133 can be configured to passively permit fluid flow thereabout when fluid is present and/or based on a given configuration of the delivery device. For example, fluid can flow past the valve 133 and can form an injectable material (e.g., an injectable hydrogel) distally from the valve 133, as illustrated in FIG. 8A.

However, the valve can be configured to block fluid flow along a flow path extending through the common branch 118c (e.g., through a common lumen in the common branch 118c), as illustrated in FIG. 8B. In some instances, a clog (represented as element 137) can be manifested within the common branch 118c, for instance. The valve 133 can be configured to passively move responsive to the presence of fluid flow and/or responsive to a chance in a configuration of the delivery device. For instance, the valve 133 has moved from the first position, illustrated in FIG. 8A, to the second position, as illustrated in FIG. 8B, responsive to the presence of the clog 137. Alternatively or in addition, the valve 133 can be configured to move to the second position responsive to a change in a configuration of the delivery device. For instance, a configuration of the delivery device can be changed responsive to pausing delivery or injection of an injectable material and can thereby promote aspects herein such as replacing a static mixer, etc.

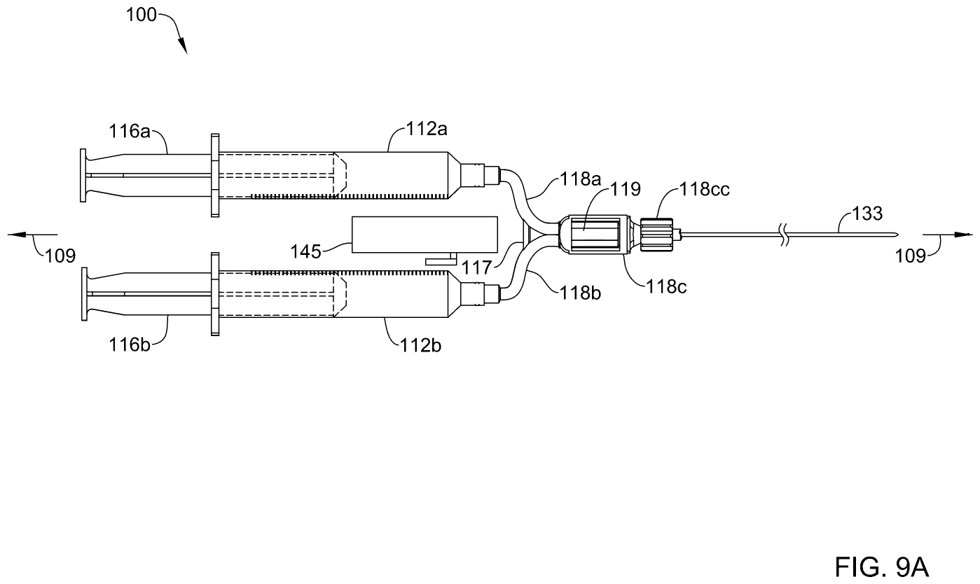

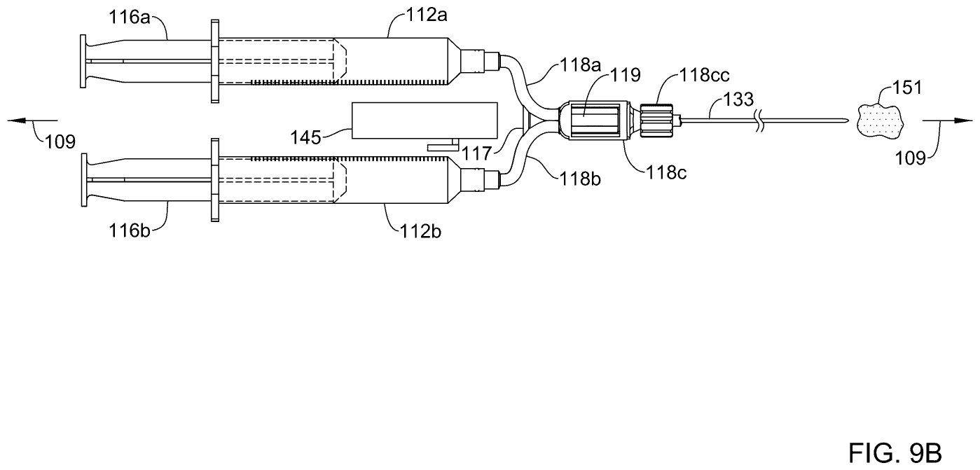

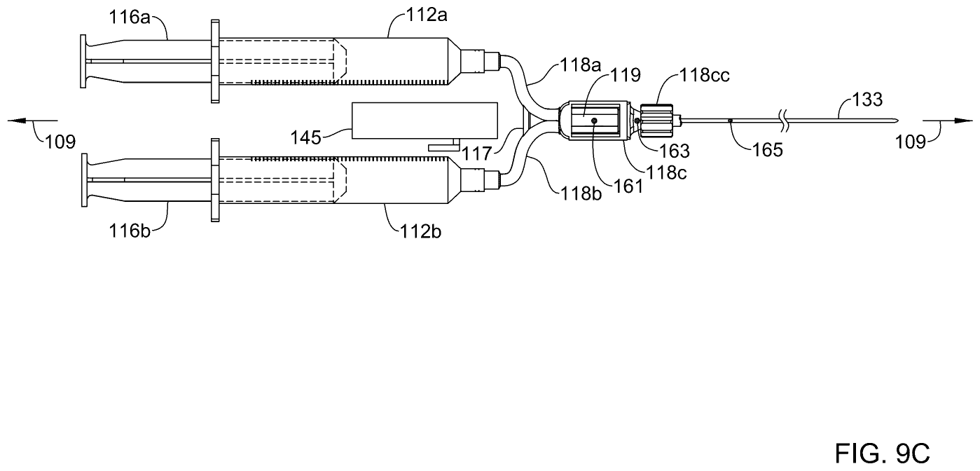

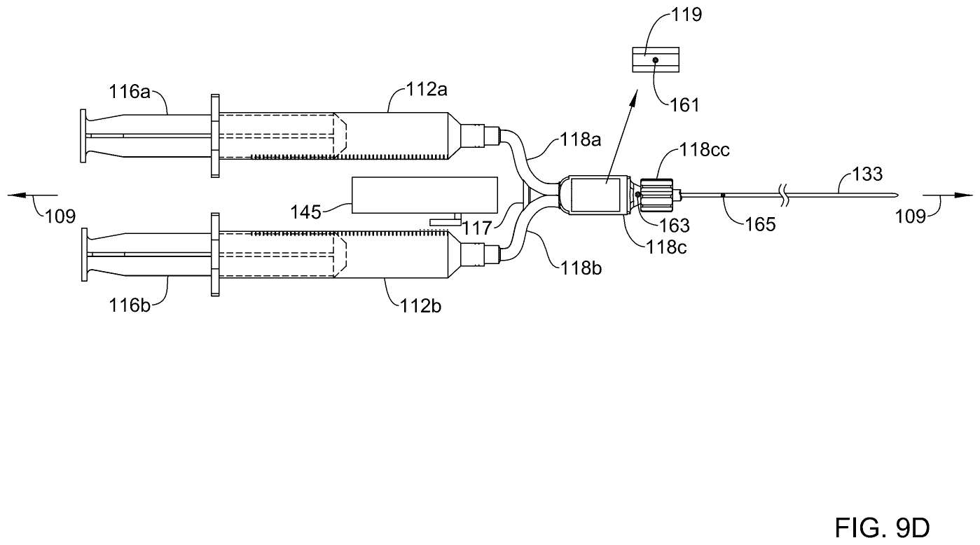

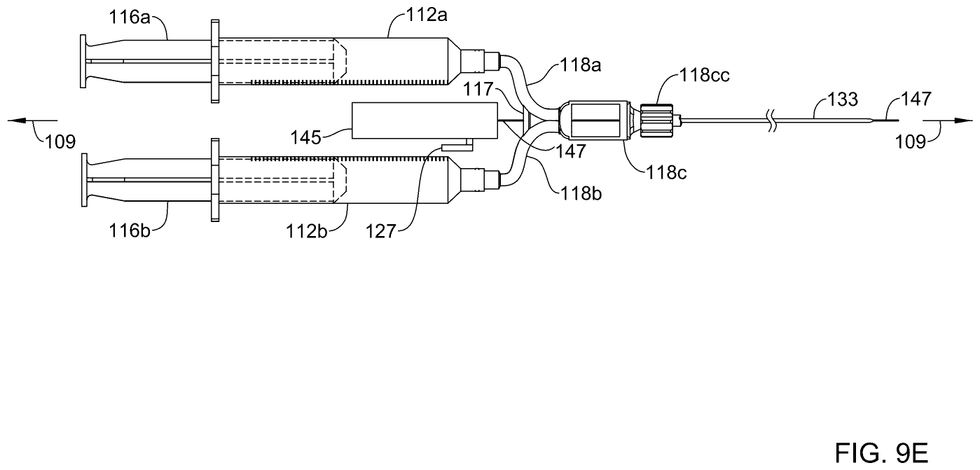

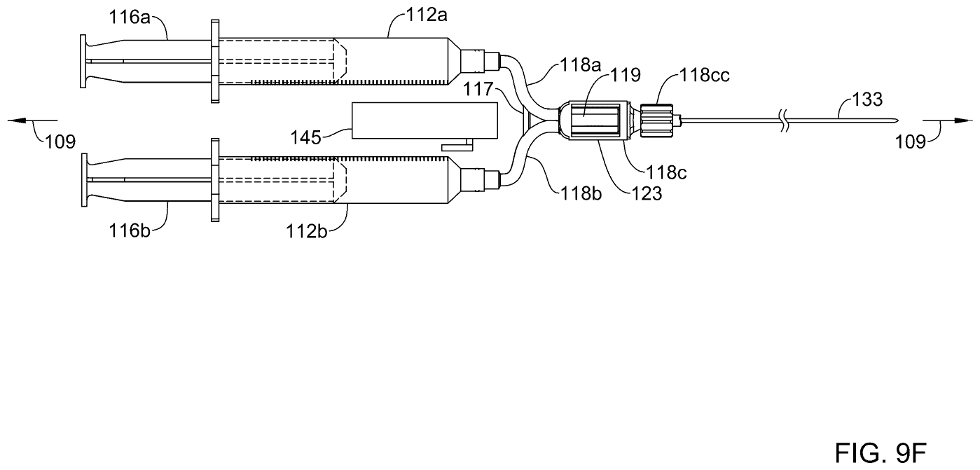

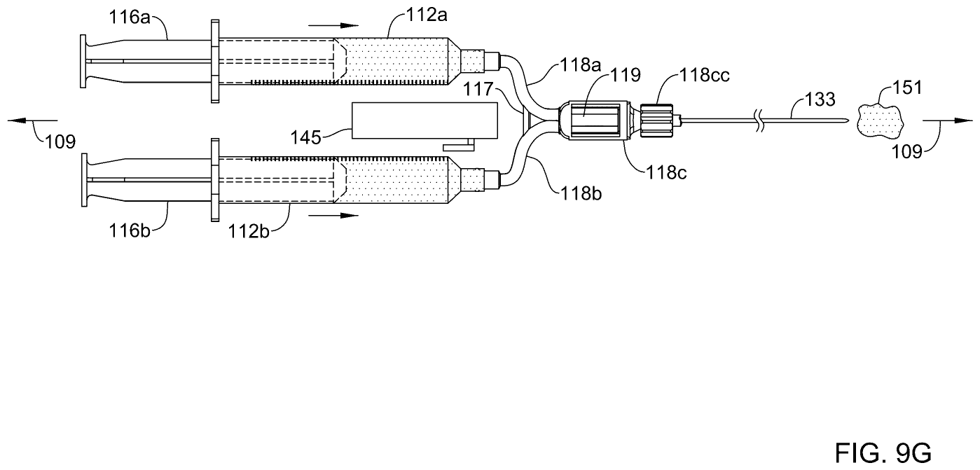

FIG. 9A schematically illustrates a delivery device 100 in a first (initial) configuration prior to injection of any hydrogel. The delivery device 100 can be analogous or similar to those previously discussed herein. In the illustrated embodiment, the clearance instrument can be manifested as a wire disposed in the wire storage device 145. The wire storage device 145 can be coupled to the delivery device 100, in some instances. Though other clearance instruments (e.g., elongate rods) can be employed and/or the clearance instrument can be housed within (e.g., within a recess in a housing) of the delivery device, in some embodiments.

FIG. 9B schematically illustrates a delivery device in a second configuration. In FIG. 6B, an individual such as an operator (e.g., a physician) begins an insertion procedure. Constituents are pushed through the syringes, mixing within static mixer, and being extruded from the needle and forming a solidified hydrogel (represented as element 151), though no clogs are present within the delivery device.

FIG. 9C schematically illustrates a delivery device in a third configuration, in accordance with an embodiment of the present disclosure. In this configuration, the operator stops during the insertion procedure. The gel polymerizes and forms clogs within the static mixer (clog #1, represented as element 161) and the connection piece between the needle and y-connector (clog #2, represented as element 163). A third clog (clog #3, represented as element 165) can be present in the needle. While three potential clog locations are illustrated, it is understood that clogs may occur anywhere along a flow path of the delivery device. Due to the presence of one or more of the clogs along a flow path of the delivery device, the operator (e.g., a physician) can no longer insert gel into a patient.

FIG. 9D schematically illustrates a delivery device in a fourth configuration, in accordance with an embodiment of the present disclosure. In this configuration, the static mixer module 119 can be removed from the common access port 123. Thus, any clogs (e.g., clog 161) that is present in the static mixer module 119 can be removed from the delivery device 100. While various Figures herein describe the replacement of a static mixer by way of removing of a static mixer module, the present disclosure is not so limited. Rather as mentioned, in some embodiments a static mixer can be replaced without removal of a static mixer module (e.g., a different static mixer from a plurality of static mixers can be moved and placed in the flow path) and/or a flow path can be altered to flow through a different static mixer of a plurality of static mixers, as described herein.

FIG. 9E schematically illustrates a delivery device in a fifth configuration, in accordance with an embodiment of the present disclosure. In this configuration the wire 147 can extend distally along the flow path to dislodge any clogs. For instance, the wire 147 can have a length such that at least a portion of the wire can extend through at least a portion of the flow path of the delivery device thereby cause any clogs (e.g., clog 163 and/or clog 165) that are located along the flow path to be ejected from the delivery device. For example, as illustrated in FIG. 9E the wire 147 can protrude distally from a distal end of the delivery device and thereby cause any clogs (e.g., clog 163 and/or clog 165) that are located along the flow path to be ejected from the delivery device. However, in some instances the wire can extend distally a portion of but not all of a length of the delivery device and thus does not protrude distally from the distal end of the delivery device (e.g., such that the wire 147) does not contact tissue or another object that is distal to the distal end of the delivery device. In any case, extending the wire 147 or another type of clearance instrument along at least a portion of the fluid path of the delivery device can restore patency of the fluid path of the delivery device.

FIG. 9F schematically illustrates a delivery device in a sixth configuration, in accordance with an embodiment of the present disclosure. In this configuration, the static mixer module 119 contains a new static mixer and the static mixer module 119 including the new static mixer has been replaced in the common access port 123 and the wire 147 has been withdrawn (fully retracted) from the delivery device into the clearance instrument storage device.

FIG. 9G schematically illustrates a delivery device in an eighth configuration, in accordance with an embodiment of the present disclosure. In this embodiment, the hydrogel injection can resume. For instance, a remaining amount of fluid in the syringes can be injected to form hydrogel (e.g., as represented by element 151).

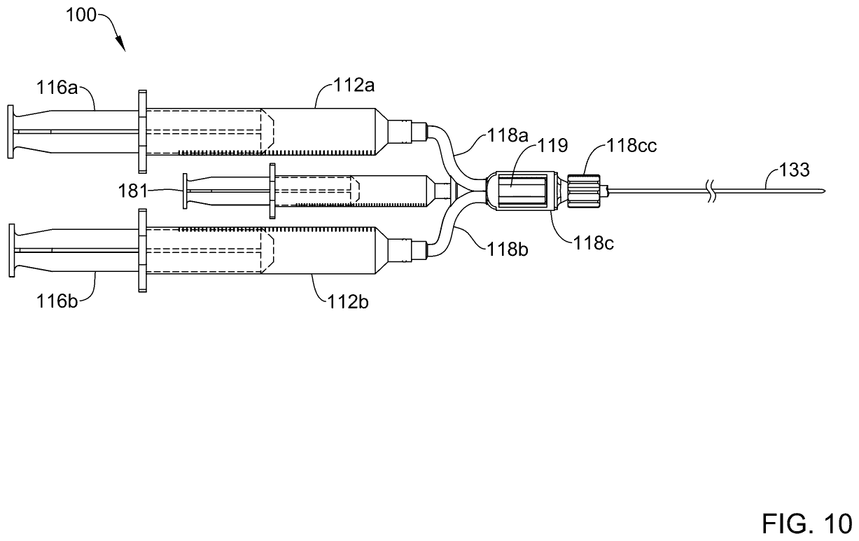

FIG. 10 schematically illustrates a delivery device including a fluid reservoir manifested in the form of a syringe 181, in accordance with an embodiment of the present disclosure. That is, in some embodiments the delivery device further comprises a third syringe comprising a third plunger and a third syringe barrel, wherein the third syringe includes a third fluid, and wherein the third syringe is fluidically coupled to the proximal access port. However, other types of reservoirs may be employed in addition to or in place of the syringe 181. The fluid may be provided in the form of ultrapure water, water for injection, saline, phosphate buffered saline, or high-ion-content water, among other types of fluids such as anesthetics fluids, etc. For instance, the fluid may be saline. The reservoir of the syringe 181 can be fluidically coupled to the proximal access port 117, as illustrated in FIG. 10. Thus, the fluid in the reservoir of the syringe can be injected or caused to flow (e.g., passively introduced by way of a pressure differential imparted by a vent or other mechanism) into the flow path of the delivery device 100 to promote aspects herein. For instance, the fluid in the reservoir of the syringe 181 can be injected into the flow path of the delivery device 100 prior to, during, and/or subsequent to hydrogel injection (e.g., prior to injection of the fluids in the syringes 112a and 112b) to minimize or eliminate the introduction of air into the flow path of the delivery device 100. For example, the fluid in the reservoir may be injected at least prior to resuming hydrogel injection utilizing a remaining amount of solution in the syringes 112a and 112b and after dislodging any clogs along the flow path of the delivery device 100, among other possibilities.

The material injection systems and kits herein can include a delivery device and a clearance instrument. However, in some embodiments one or more additional components (e.g., sensing components and/or sterile water reservoirs can be provided).

The clearance instrument can be an elongate rod, a wire, a flexible rail, tool, barbed shaft, auger, corkscrew, skewer, bore snake, lumen snake, plunger, obturator, clog removal tool, flexible barbed, collapsible clog retention barbs, and/or other type of clearance instrument.

In some embodiments, the clearance instrument can be stored in a clearance instrument storage device, and the clearance instrument storage device can be coupled to or is removably coupled to the delivery device. For instance, the clearance instrument storage device can be coupled to or included in (e.g., formed of a cavity within) a syringe holder of the delivery device, among other possibilities.

In some embodiments the clearance instrument can be an elongate rod or a wire. For instance, the clearance instrument can be a rigid or substantially rigid elongate rod. Employing a rigid or substantially rigid elongate rod can promote aspects herein such as promoting dislodging of any clogs formed in the delivery device 100. FIG. 11 illustrates an elongate rod 135 disposed within the proximal access port 117 and extending longitudinally along a fluid flow path within the delivery device 100.

However, in some embodiments the clearance instrument can be a flexible or relatively bendable wire. For instance, in some embodiments, the clearance instrument can be a wire. In such embodiments, the wire can be manifested as a coiled wire such as a coiled wire that is stored in a clearance instrument storage device (e.g., a box including a component (e.g., cylindrical component)) about which the wire can be coiled and/or uncoiled. In some embodiments, the clearance instrument storage device can include a mechanism such as a handle, wheel, or other type of mechanism (e.g., a spring-loaded mechanism, etc.) that can be actuated to cause a clearance instrument to extend and/or retract. For instance, the mechanism can be manifested as a handle, wheel, and/or other type of mechanism extending from an exterior surface of the clearance instrument storage device, among other possibilities. In such instances, rotation and/or otherwise actuating the mechanism can cause the wire to coil or uncoil. Actuation of the mechanism can be manual activation performed by a user or can be automatic actuation. Employing a flexible or relatively bendable clearance instrument can promote aspects herein such as promoting the clearing of clogs that are distal to the having a common branch and/or permit the insertion of the clearance instrument into an access port that not positioned along the longitudinal axis (e.g., is located coaxial with the longitudinal axis) of the delivery device 100.

Methods of material (e.g., hydrogel) delivery or injection are described herein. The methods can be employed with the injection devices described herein. For instance, in some embodiments, the method can further include simultaneously or substantially simultaneously pressing a first plunger and a second plunger into a first syringe barrel and a second syringe barrel, respectively, to cause a portion of a first solution in the first syringe and a second solution in the second syringe to form an injectable material that is delivered (e.g., injected). Notably, the methods herein can permit stopping and starting the injection of material. For instance, the method can include pausing pressing (e.g., pausing an application of pressure) the first plunger and second plunger prior to injection of all of the first solution and the second solution. Subsequent to pausing the injection, the method can include dislodging, via a peripheral access port, a common access port, or both, a clog formed of a portion of the injectable material that remains along an injection path of an injection device.

In some embodiments, the method includes dislodging the clog by passing a clearance instrument through an access port. As used herein, “passing” refers to the insertion (e.g., when the clearance instrument is not present in or adjacent to a flow path of the delivery device during material injection) and/or removal of the clearance instrument from a flow path of the delivery device (e.g., when the clearance instrument is present in or adjacent to a flow path of the delivery device during material injection). In any case, the clearance instrument can be passed through at least a portion of the flow path of the delivery device. For instance, as mentioned the clearance instrument can be passed through a portion of but not all of the flow path of the delivery device to avoid contacting any tissue or another object that is located distal to a distal tip of the delivery devices, as described herein. The access port can be a proximal access port and/or a bypass access port. For instance, the method can include dislodging the clog by passing a clearance instrument through the proximal access port into at least the common lumen. For instance, the method can include passing an elongate rod, via a proximal insertion port, through the proximal access port into the common lumen and through at least a portion of a lumen of needle or the tube (e.g., to cause a portion of the elongate rod to extend distally through at least a portion of the needle or tube) to dislodge any clogs located along an entire material flow path extending through the common lumen and the lumen of the needle or tube. In some embodiments, a method of hydrogel injection can comprise connecting a first syringe barrel of a first syringe to a first branch lumen of the Y-connector; connecting a second syringe barrel of the second syringe to a second branch lumen of the Y-connector; connecting a needle or tube to the common lumen of the Y-connector to form a delivery device. The method can further include simultaneously or substantially simultaneously pressing the first plunger and the second plunger into the first syringe barrel and the second syringe barrel, respectively, to cause a portion of first solution in the first syringe and a second solution in the second syringe to form an injectable hydrogel that is injected. Notably, the methods herein can permit stopping and starting the injection of hydrogel. For instance, the method can include pausing pressing (e.g., pausing an application of pressure) the first plunger and second plunger prior to injection of all of the first solution and the second solution. Subsequent to pausing the injection, the method can include dislodging, via a peripheral access port, a common access port, or both, a clog formed of a portion of the injectable hydrogel that remains along an injection path of the hydrogel injection device. In some embodiments, the method includes dislodging the clog by passing a clearance instrument through the proximal access port into at least the common lumen. For instance, the method can include passing an elongate rod, via a proximal insertion port, through the common lumen and through at least a portion of a lumen of needle or the tube (e.g., to cause a portion of the elongate rod to extend along at least a portion of the flow path of the delivery device) to dislodge any clogs located along at least a portion of the flow path extending through the common lumen and the lumen of the needle or tube. For example, the clearance instrument can extend to, but does not extend distally past, a distal end of the delivery device, among other possibilities.

In some embodiments, the method can include removing a plug from the peripheral access port prior to passing a clearance instrument through the proximal access port to dislodge the clog. In such embodiments, the method can include replacing the plug in the peripheral access port prior to simultaneously pressing the first plunger and the second plunger into the first syringe barrel and the second syringe barrel, respectively, to cause at least part of a remaining portion of first solution in the first syringe and a second solution in the second syringe.

In some embodiments, the method can include removing the removable mixer module from the delivery device (e.g., removing the removable mixer module) from the common access port prior to passing a clearance instrument through the proximal access port to dislodge the clog. In such embodiments, the method can include replacing the removable mixer module the common access port prior to simultaneously pressing the first plunger and the second plunger into the first syringe barrel and the second syringe barrel, respectively, to cause at least part of a remaining portion of first solution in the first syringe and a second solution in the second syringe.

In some embodiments, the method can include removing a plug from the peripheral access port and removing the removable mixer module from the common access port prior to passing a clearance instrument through the proximal access port to dislodge the clog. In such embodiments, the method can include replacing the plug in the peripheral access port and replacing the removable mixer module in the common access port prior to simultaneously pressing the first plunger and the second plunger into the first syringe barrel and the second syringe barrel, respectively, to cause at least part of a remaining portion of first solution in the first syringe and a second solution in the second syringe.

Subsequent to dislodging the clog, the method can include simultaneously pressing the first plunger and the second plunger into the first syringe barrel and the second syringe barrel, respectively, to cause at least part of a remaining portion of first solution in the first syringe and a second solution in the second syringe to form an injectable hydrogel that is injected.

In some embodiments, the method can include replacing a static mixer in the delivery device (e.g., a hydrogel injection device) prior to simultaneously pressing the first plunger and the second plunger into the first syringe barrel and the second syringe barrel, respectively, to cause at least part of a remaining portion of first solution in the first syringe and a second solution in the second syringe. The static mixer can be replaced with a new (unused and clog-free) static mixer. In some embodiments, the replacement (e.g., new) static mixer can be placed in a removable mixer module, and the removable mixer module can then be reinserted into the injection device. However, in some embodiments, the replacement (e.g., new) static mixer can be included in a plurality of static mixers that are coupled to delivery device (e.g., coupled to the common branch thereof) and that are movable relative to the common lumen of the delivery device, as described herein. In some embodiments, the method can include dislodging the clog while a removable mixer module is removed from a delivery device and/or is moved at least partially outside a hydrogel flow path extending along the common lumen of the delivery device.

The systems and kits described herein may be used for in a variety of medical procedures and/or non-medical procedures. For example, the systems and kits of the present disclosure may be used to provide fiducial markers, to provide tissue augmentation or regeneration, to provide a filler or replacement for soft tissue, to provide mechanical support for compromised tissue, to provide a scaffold, as a carrier of therapeutic agents in the treatment of diseases and cancers and the repair and regeneration of tissue, among other uses.

The systems and kits of the present disclosure may be used in a variety of medical procedures, including the following, among others: a procedure to implant a fiducial marker comprising a hydrogel, a procedure to implant a tissue regeneration scaffold comprising a hydrogel, a procedure to implant a tissue support comprising a hydrogel, a procedure to implant a tissue bulking agent comprising a hydrogel, a procedure to implant a therapeutic-agent-releasing depot comprising a hydrogel, a tissue augmentation procedure comprising implanting a hydrogel, a procedure to introduce a hydrogel between a first tissue and a second tissue to space the first tissue from the second tissue.

The systems and kits of the present disclosure may be used in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for A sherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Although various embodiments are specifically illustrated and described herein, it will be appreciated that modifications and variations of the present disclosure are covered by the above teachings and are within the purview of any appended claims without departing from the spirit and intended scope of the present disclosure.

What is claimed is:

1. A delivery device comprising:
a first syringe comprising a first plunger and a first syringe barrel that terminates in a first connector and,
a second syringe comprising a second plunger and a second syringe barrel that terminates in a second connector;
a needle or a tube; and
a Y-connector, wherein the Y-connector includes at least one access port.

2. The delivery device of claim 1, wherein the at least one access port includes a proximal access port or a common access port.

3. The delivery device of claim 1, wherein the at least one access port includes both a proximal access port and a common access port.

4. The delivery device of claim 3, wherein the proximal access port is located at a proximal end of a common branch of the Y-connector.

5. The delivery device of claim 4, wherein at least a portion of the proximal access port is located along a longitudinal axis of the delivery device.

6. The delivery device of claim 4, wherein the proximal access port is configured to permit a clearance instrument to extend through the proximal access port at least into a common lumen of the common branch.

7. The delivery device of claim 3, wherein at least a portion of the common access port is located along a longitudinal axis of the Y-connector.

8. The delivery device of claim 7, wherein the common access port is an elongate recess in a common branch of the Y-connector, and wherein the elongate recess is configured to receive a removable mixer module.

9. The delivery device of claim 8, further comprising a removable mixer module that is removably disposed in the common access port.

10. The delivery device of claim 9, wherein the removable mixer module includes a handle.

11. The delivery device of claim 1, wherein the delivery device includes a movable mixer module including a plurality of static mixers coupled thereto, wherein the movable mixer module is configured to position an individual static mixer, of the plurality of static mixers, in a common lumen of a common branch of the Y-connector.

12. The delivery device of claim 11, wherein the movable mixer module is movable between a first position and a second position, and wherein a first static mixer of the plurality of static mixers is in the common lumen when the movable mixer module is in the first position, and wherein a second static mixer of the plurality of static mixer is in the common lumen when the movable mixer module is in the second position.

13. The delivery device of claim 1, wherein the at least one access port includes a proximal access port, wherein the delivery device further comprises a third syringe comprising a third plunger and a third syringe barrel, and wherein the third syringe is fluidically coupled to the proximal access port.

14. The delivery device of claim 13, wherein the third syringe includes a fluid.

15. The delivery device of claim 1, wherein the delivery device includes a plurality of static mixers that are fixed relative to the delivery device, and wherein the delivery device is configured to:
select a first static mixer for a flow path of the delivery device extend therethrough in a first configuration; and
select a second static mixer for the flow path of the delivery device extend therethrough in a second configuration.

16. A material injection system comprising;
a delivery device comprising:
a first syringe comprising a first plunger and a first syringe barrel that terminates in a first connector;
a second syringe comprising a second plunger and a second syringe barrel that terminates in a second connector;
a needle or a tube; and
a Y-connector that comprises a first branch lumen having a first end and a second end, a second branch lumen having a first end and a second end, and a common branch that comprises a common lumen having a first end and a second end, the first end of the first branch lumen and the first end of the second branch lumen in fluid communication with the first end of the common lumen, the second end of the first branch lumen terminating at a connector which is configured to connect with the first connector, the second end of the second branch lumen terminating at a connector which is configured to connect with the second connector, and the second end of the common lumen terminating at a connector configured to connect to a connector of the needle or the tube, wherein the Y-connector includes at least one access port; and
a clearance instrument.

17. The material injection system of claim 16, wherein the clearance instrument comprises an elongate rod or a wire.

18. The material injection system of claim 16, wherein the clearance instrument is stored in a clearance instrument storage device, and wherein the clearance instrument storage device is coupled to or is removably coupled to the delivery device.

19. A method of material injection, comprising:
simultaneously pressing a first plunger and a second plunger into a first syringe barrel of a first syringe and a second syringe barrel of a second syringe, respectively, to cause a portion of a first solution in the first syringe and a second solution in the second syringe to form an injectable material that is injected;
pausing pressing the first plunger and second plunger prior to injection of all of the first solution and the second solution;
dislodging, via a peripheral access port, a common access port, a bypass port, or any combination thereof, a clog formed of a portion of the injectable material that remains along an injection path of a delivery device, wherein dislodging the clog comprises passing a clearance instrument through the peripheral access port, the common access port, the bypass port, or any combination thereof, along at least a portion of a flow path of the delivery device; and
simultaneously pressing the first plunger and the second plunger into the first syringe barrel and the second syringe barrel, respectively, to cause at least part of a remaining portion of the first solution in the first syringe and at least part of the second solution in the second syringe to form an injectable material.

20. The method of claim 19, wherein the injectable material is an injectable hydrogel.
